(19) United States
(12) Patent Application Publication
(10) Pub. No.: US 2025/0302784 A1
(43) Pub. Date: Oct. 2, 2025

(54) MYOBLAST PROLIFERATION PROMOTER AND MYOBLAST-PROLIFERATION PROMOTING COMPOSITION, AND MUSCLE ATROPHY INHIBITOR AND MUSCLE-ATROPHY INHIBITING COMPOSITION

(71) Applicant: MARUZEN PHARMACEUTICALS CO., LTD., Hiroshima (JP)

(72) Inventors: Susumu YOSHINO, Hiroshima (JP); Hiroyasu IWAHASHI, Hiroshima (JP)

(21) Appl. No.: 18/865,847

(22) PCT Filed: Apr. 14, 2023
(86) PCT No.: PCT/JP2023/015250

§ 371 Date: Nov. 14, 2024

(30) Foreign Application Priority Data
Jun. 13, 2022 (JP) 2022-095282

(51) Int. Cl. A61K 31/192; A61P 21/00

(52) U.S. Cl. CPC A61K 31/192; A61P 21/00

(57) ABSTRACT

A myoblast proliferation promoter and a muscle atrophy inhibitor are provided, each of which includes a compound represented by Structural Formula (1) below,

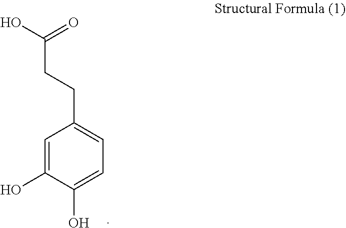 [SMILES: O=C(O)CCc1ccc(O)c(O)c1]

### TECHNICAL FIELD

The present invention relates to myoblast proliferation promoters and myoblast-proliferation promoting compositions, and muscle atrophy inhibitors and muscle-atrophy inhibiting compositions.

### BACKGROUND ART

In Japan, the aging population rate (the proportion of the population aged 65 years old or older) has been increasing year by year. In the aging society, it is important to improve the quality of life and extend healthy life expectancy.

Muscle atrophy refers to muscle thinning. For example, it has been known that disuse of muscles due to a sedentary lifestyle or bed rest causes disuse muscle atrophy, and aging also causes age-related muscle atrophy due to loss of homeostasis of physiological tissues of muscles.

Such functional decline in muscles leads to a shortage of exercise or activities, and mental stress, and may further impair other physical functions. Thus, quality of life may be impaired, or the healthy life may be shortened.

Accordingly, it is extremely important to prevent the functional decline of muscles.

As a composition having an excellent effect of maintaining muscle fibers constituting muscles, a composition for maintaining muscle fibers, which includes a theaflavin compound as an active ingredient, has been proposed (see, for example, Patent Document 1).

However, there is still a strong demand for a novel material, which can prevent functional decline of muscles, is highly safe, and can be therefore widely used as a substance for food and drink products, pharmaceutical products, research reagents, and the like.

### CITATION LIST

### Patent Documents

Patent Document 1: Japanese Unexamined Patent Application Publication No. 2021-169430

### SUMMARY OF THE INVENTION

### Technical Problem

The present invention aims to solve the various problems existing in the related art, and achieve the following objects. Specifically, the object of the present invention is to provide a myoblast proliferation promoter and a myoblast-proliferation promoting composition, which have an excellent myoblast-proliferation promotion effect and are highly safe.

Moreover, another object of the present invention is to provide a muscle atrophy inhibitor and a muscle-atrophy inhibiting composition, which have an excellent muscle-atrophy inhibition effect and are highly safe.

### Solution to the Problem

The present inventors have conducted research to solve the above problems. As a result, the present inventors have reached the insight such that a compound represented by Structural Formula (1) below has an excellent myoblast-proliferation promotion effect and an excellent muscle-atrophy inhibition effect, is highly safe, and is effective for myoblast proliferation promotion and muscle atrophy inhibition.

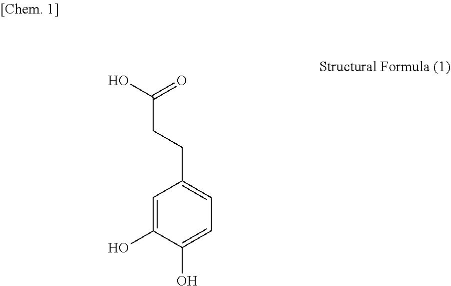 [SMILES: O=C(O)CCc1ccc(O)c(O)c1]

The present invention is based on the above insight of the present inventors, and the means for solving the above problems are as follows.

<1> A myoblast proliferation promoter includes a compound represented by Structural Formula (1) below,

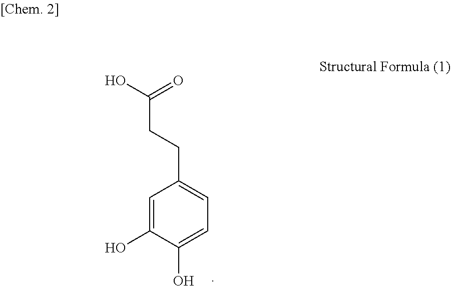 [SMILES: O=C(O)CCc1ccc(O)c(O)c1]

<2> A myoblast-proliferation promoting composition includes the myoblast proliferation promoter according to <1>.

<3> A muscle atrophy inhibitor includes a compound represented by Structural Formula (1) below,

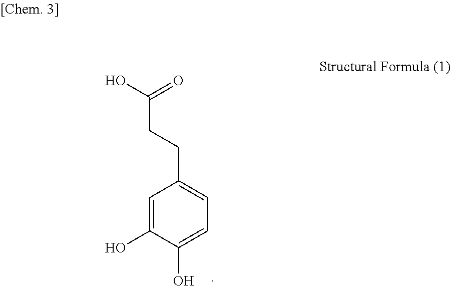 [SMILES: O=C(O)CCc1ccc(O)c(O)c1]

<4> A muscle-atrophy inhibiting composition includes the muscle atrophy inhibitor according to <3>.

### Effects of the Invention

According to the myoblast proliferation promoter and myoblast-proliferation promoting composition of the present invention, the various problems existing in the related art can be solved, the above object can be achieved, and a myoblast proliferation promoter and a myoblast-proliferation promoting composition, which have an excellent myoblast-proliferation promotion effect and are highly safe, can be provided.

According to the muscle atrophy inhibitor and muscle-atrophy inhibiting composition of the present invention, the various problems existing in the related art can be solved, the above object can be achieved, and a muscle atrophy inhibitor and a muscle-atrophy inhibiting composition, which have an excellent muscle-atrophy inhibition effect and are highly safe, can be provided.

### DESCRIPTION OF EMBODIMENTS

### (Myoblast Proliferation Promoter and Muscle Atrophy Inhibitor)

The myoblast proliferation promoter of the present invention includes a compound represented by Structural Formula (1) below as an active ingredient, and may further include other ingredients as necessary.

The muscle atrophy inhibitor of the present invention includes a compound represented by Structural Formula (1) below as an active ingredient, and may further include other ingredients as necessary.

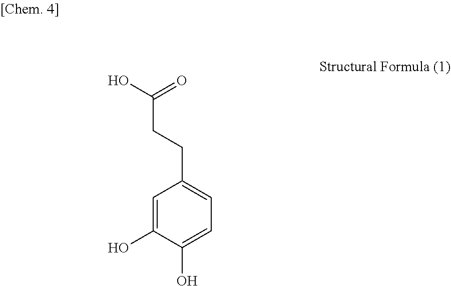 [SMILES: O=C(O)CCc1ccc(O)c(O)c1]

In the present specification, the myoblast proliferation promotion means promotion of proliferation of myoblasts. The degree of the proliferation is not particularly limited, and may be appropriately selected according to the intended purpose.

A variety of the myoblasts is not particularly limited, and may be appropriately selected according to the intended purpose. Examples of the variety include myoblasts derived from skeletal muscles and the like.

The origin of the myoblasts is not particularly limited, and may be appropriately selected according to the intended purpose. Examples of the origin include humans, animals other than humans (e.g., mice, rats, hamsters, dogs, cats, cows, pigs, monkeys, etc.), and the like.

In the present specification, the muscle atrophy inhibition means inhibition of a muscle from becoming thinner. Specifically, the muscle atrophy inhibition encompasses inhibition of reduction in a muscle mass, prevention of suppressed muscle-mass increase, or the like. The degree of the muscle atrophy inhibition is not particularly limited, and may be appropriately selected according to the intended purpose.

A variety of the muscle atrophy is not particularly limited, and may be appropriately selected according to the intended purpose. Examples of the variety include disuse muscle atrophy, age-related muscle atrophy, and the like.

The fact that the compound represented by Structural Formula (1) has an excellent myoblast-proliferation promotion effect and an excellent muscle-atrophy inhibition effect and is effective as a myoblast proliferation promoter or a muscle atrophy inhibitor has not been known at all in the related art and is a novel insight of the present inventors.

### <Compound Represented by Structural Formula (1)>

The name of the compound represented by Structural Formula (1) below is 3,4-dihydroxyhydrocinnamic acid.

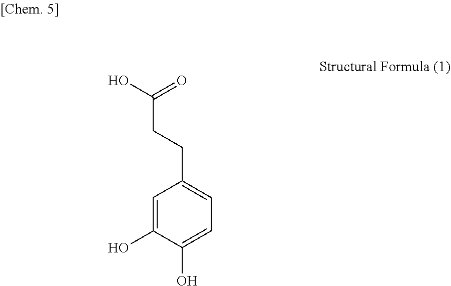 [SMILES: O=C(O)CCc1ccc(O)c(O)c1]

The compound represented by Structural Formula (1) is a known compound. As the compound represented by Structural Formula (1), a commercially available product may be used, or a compound extracted from plants or the like may be used.

Since the compound represented by Structural Formula (1) above has an excellent myoblast-proliferation promotion effect and an excellent muscle-atrophy inhibition effect, the compound represented by Structural Formula (1) can be used as an active ingredient of the myoblast proliferation promoter or the muscle atrophy inhibitor. The myoblast proliferation promoter and muscle atrophy inhibitor can be used in a variety of use, such as pharmaceutical products, quasi-drugs, oral compositions, cosmetic products, reagents for research, and the like.

Each of the myoblast proliferation promoter and the muscle atrophy inhibitor may be composed solely of the compound represented by Structural Formula (1), or may be a preparation including the compound represented by Structural Formula (1).

Each of the myoblast proliferation promoter and the muscle atrophy inhibitor can be formulated into any dosage form, such as powders, granules, tablets, liquids, or the like, using a pharmaceutically acceptable carrier, such as dextrin, cyclodextrin, or the like, or any other auxiliary agents according to a common method. As the auxiliary agents, for example, excipients, binders, disintegrating agents, lubricants, stabilizers, flavoring agents, or the like can be used. Each of the myoblast proliferation promoter and the muscle atrophy inhibitor can be used by being blended in another composition (e.g., the below-described myoblast-proliferation promoting composition, muscle-atrophy inhibiting composition, etc.), and can be used as tablets, capsules, or the like.

An amount of the compound represented by Structural Formula (1) in the preparation of the myoblast proliferation promoter or the preparation of the muscle atrophy inhibitor is not particularly limited, and may be appropriately selected according to the intended purpose.

Note that the myoblast proliferation promoter may use, as necessary, another ingredient having a myoblast-proliferation promotion effect together with the compound represented by Structural Formula (1) as active ingredients.

Moreover, the muscle atrophy inhibitor may use, as necessary, another ingredient having a muscle-atrophy inhibition effect together with the compound represented by Structural Formula (1) as active ingredients.

### <Other Ingredients>

Other ingredients are not particularly limited as long as the effects obtainable by the present invention are not adversely affected, and may be appropriately selected according to a usage form of the myoblast proliferation promoter or the muscle atrophy inhibitor. Examples of the above other ingredients include the above-described ingredients used for formulating the preparations. Such ingredients may be used alone or in combination.

An amount of the other ingredients in the myoblast proliferation promoter or the muscle atrophy inhibitor is not particularly limited, and may be appropriately selected according to the intended purpose.

An administration method of each of the myoblast proliferation promoter and the muscle atrophy inhibitor is not particularly limited, and may be appropriately selected according to the intended purpose. Examples of the administration method include oral administration and the like.

An administration dose, an administration period, and an administration interval of each of the myoblast proliferation promoter and the muscle atrophy inhibitor is not particularly limited, and may be appropriately selected according to the intended purpose.

### <Use>

Use of the myoblast proliferation promoter is not particularly limited, and may be appropriately selected according to the intended purpose. For example, the myoblast proliferation promoter can be suitably used as an active ingredient of the below-described myoblast-proliferation promoting composition.

Use of the muscle atrophy inhibitor is not particularly limited, and may be appropriately selected according to the intended purpose. For example, the muscle atrophy inhibitor is suitably used as an active ingredient of the below-described muscle-atrophy inhibiting composition.

The myoblast proliferation promoter can promote proliferation of myoblasts through the action of the compound represented by Structural Formula (1) serving as an active ingredient. Thus, the myoblast proliferation promoter of the present invention can be used in any application in which a myoblast-proliferation promotion effect is beneficial.

The muscle atrophy inhibitor can inhibit muscle atrophy through the action of the compound represented by Structural Formula (1) serving as an active ingredient. Thus, the muscle atrophy inhibitor of the present invention can be used in any application in which a muscle-atrophy inhibition effect is beneficial.

Since the myoblast proliferation promoter has an excellent myoblast-proliferation promotion effect and the muscle atrophy inhibitor has an excellent muscle-atrophy inhibition effect, the myoblast proliferation promoter or the muscle atrophy inhibitor can be blended in, for example, an oral composition, such as food and drink products. In this case, the compound represented by Structural Formula (1) may be blended in the oral composition as it is, or the compound represented by Structural Formula (1) is formulated as a preparation of the myoblast proliferation promoter or the muscle atrophy inhibitor, and the preparation may be blended in the oral composition.

The myoblast proliferation promoter and muscle atrophy inhibitor of the present invention are suitably applied to humans, but can be applied to animals other than humans (e.g., mice, rats, hamsters, dogs, cats, cows, pigs, monkeys, etc.) as long as the functions and effects of the myoblast proliferation promoter and muscle atrophy inhibitor can be exhibited.

Usage of each of the myoblast proliferation promoter and the muscle atrophy inhibitor is not particularly limited, and may be appropriately selected according to the intended purpose. Examples of the usage include oral administration, parenteral administration, external application, and the like.

A usage site, usage method, and dosage form of each of the myoblast proliferation promoter and the muscle atrophy inhibitor can be appropriately selected according to the intended use and purpose.

A usage method, such as a usage amount, a usage period, a usage interval, and the like, of each of the myoblast proliferation promoter and the muscle atrophy inhibitor is not particularly limited, and may be appropriately selected according to the intended purpose.

Moreover, the myoblast proliferation promoter of the present invention can be also used as a reagent for research associated with an action mechanism of the myoblast-proliferation promotion effect; the muscle atrophy inhibitor of the present invention can be also used as a reagent for research associated with an action mechanism of the muscle-atrophy inhibition effect.

### (Myoblast-Proliferation Promoting Composition and Muscle-Atrophy Inhibiting Composition)

The myoblast-proliferation promoting composition of the present invention includes the myoblast proliferation promoter of the present invention, and may further include other ingredients as necessary.

The muscle-atrophy inhibiting composition of the present invention includes the muscle atrophy inhibitor of the present invention, and may further include other ingredients as necessary.

### <Myoblast Proliferation Promoter and Muscle Atrophy Inhibitor>

The myoblast proliferation promoter is the above-described myoblast proliferation promoter of the present invention.

The muscle atrophy inhibitor is the above-described muscle atrophy inhibitor of the present invention.

An amount of the myoblast proliferation promoter in the myoblast-proliferation promoting composition is not particularly limited, and may be appropriately adjusted according to a form of the myoblast-proliferation promoting composition. The amount of the myoblast proliferation promoter, which is determined by converting into an amount of the compound represented by Structural Formula (1), is preferably from 0.0001% by mass to 20% by mass, and more preferably from 0.0001% by mass to 10% by mass. The myoblast-proliferation promoting composition may be composed solely of the myoblast proliferation promoter.

An amount of the muscle atrophy inhibitor in the muscle-atrophy inhibiting composition is not particularly limited, and may be appropriately adjusted according to a form of the muscle-atrophy inhibiting composition or the like. The amount of the muscle atrophy inhibitor, which is determined by converting into the amount of the compound represented by Structural Formula (1), is preferably from 0.0001% by mass to 20% by mass, and more preferably from 0.0001% by mass to 10% by mass. The muscle-atrophy inhibiting composition may be composed solely of the muscle atrophy inhibitor.

### <Other Ingredients>

Other ingredients in each of the myoblast-proliferation promoting composition and the muscle-atrophy inhibiting composition are not particularly limited, and may be appropriately selected according to a usage form of the myoblast-proliferation promoting composition or the muscle-atrophy inhibiting composition. Examples of the above other ingredients include the same ingredients as the other ingredients described in the above section of the myoblast proliferation promoter and muscle atrophy inhibitor. Such ingredients may be used alone or in combination.

An amount of the other ingredients in the myoblast-proliferation promoting composition or the muscle-atrophy inhibiting composition is not particularly limited, and may be appropriately selected according to the intended purpose.

### Embodiments

Embodiments of the myoblast-proliferation promoting composition and the muscle-atrophy inhibiting composition are not particularly limited, and may be appropriately selected according to the intended purpose. Examples of the embodiments include pharmaceutical products, quasi-drugs, food and drink products, and the like.

The myoblast-proliferation promoting composition and muscle-atrophy inhibiting composition of the present invention can be used daily, and can extremely effectively exhibit a variety of pharmacological activities including a myoblast-proliferation promotion effect and a muscle-atrophy inhibition effect through the action of the compound represented by Structural Formula (1) serving as an active ingredient.

The myoblast-proliferation promoting composition and muscle-atrophy inhibiting composition of the present invention are suitably applied to humans, but can be applied to animals other than humans (e.g., mice, rats, hamsters, dogs, cats, cows, pigs, monkeys, etc.) as long as the functions and effects of the myoblast-proliferation promoting composition and muscle-atrophy inhibiting composition can be exhibited.

Usage of the myoblast-proliferation promoting composition and muscle-atrophy inhibiting composition of the present invention are not particularly limited, and may be appropriately selected. Examples of the usage include oral administration, parenteral administration, external application, and the like. The usage is preferably oral administration.

Examples of the composition for oral administration include oral preparations, food and drink products, and the like. In the present specification, the food and drink products refer to products that have little risk for harming human health and can be consumed in ordinary social life through oral administration or gastrointestinal administration. The food and drink products are not limited to categories according to the administrative classification, such as foods, pharmaceutical products, and quasi-drugs. Accordingly, the food and drink products include a wide range of food and drink products including general food products, health foods (functional food and drink products), health-promoting food products (food for specified health uses, food with nutrient function claims, and food with functional claims), quasi-drugs, pharmaceutical products, etc., which are for oral intake.

A type of the composition for the oral administration is not particularly limited, and may be appropriately selected. Examples thereof include: beverages such as tea beverages, soft drinks, carbonated beverages, nutritional beverages, fruit-based beverages, lactic beverages, alcoholic beverages, coffee beverages, coffee-containing soft drinks, and the like (including concentrates of the foregoing beverages and powders for preparing the foregoing beverages); frozen desserts such as ice cream, sherbet, shaved ice, and the like; noodles, such as buckwheat noodles, udon noodles, starch noodles, Gyoza skins, Shumai skins, Chinese noodles, instant noodles, and the like; confectionary, such as hard candies, candies, chewing gums, chocolate, tablets, snacks, biscuits, jelly, jams, creams, sweet pastries, bread, and the like; fishery products, such as crabs, salmon, clams, tuna, sardine, shrimp, bonito, mackerel, whale, oysters, Pacific saury, squid, Anadara broughtonii, scallops, abalone, sea urchin, salmon roe, Sulculus diversicolor supertexta, and the like; fish or meat processed foods, such as fish paste, ham, sausages, and the like; dairy products, such as processed milk, fermented milk, and the like; fat or oil, or fat or oil processed food products, such as salad oil, tempura oil, margarines, mayonnaise, shortening, whipping creams, dressing, and the like; seasonings, such as sauces and dipping sauces; retort pouch-packaged foods, such as curry, stew, Oyakodon, gruel, rice soups, Chukadon, a bowl of rice with breaded pork cutlets, a bowl of rice with tempura, Unadon, Hayashi rice, Oden, Mapo tofu, a bowl of rice with beef, Bolognese sauces, egg soup, omelette made with rice, Gyoza, Shumai, hamburgers, meat balls, and the like; daily dishes, such as salads and pickles; various forms of health, cosmetic, or nutritional supplementary foods; pharmaceutical products and quasi-drugs, such as tablets, powders, capsules, granules, extracts, syrups, drink preparations, lozenges, mouthwash, and the like; oral fresheners used inside the mouth, such as mouth freshener and breath freshener; toothpaste; and the like.

A production method for each of the myoblast-proliferation promoting composition and the muscle-atrophy inhibiting composition is not particularly limited, and may be appropriately selected according to a usage form of the myoblast-proliferation promoting composition or the muscle-atrophy inhibiting composition.

A usage dose, a usage period, a usage interval and the like of each of the myoblast-proliferation promoting composition and the muscle-atrophy inhibiting composition are not particularly limited, and may be appropriately selected according to the intended purpose.

As described above, the myoblast proliferation promoter and myoblast-proliferation promoting composition of the present invention have an excellent myoblast-proliferation promotion effect.

Accordingly, the present invention also relates to a myoblast-proliferation promotion method including administration of at least one selected from the group consisting of the myoblast proliferation promoter and the myoblast-proliferation promoting composition to a subject.

As described above, the muscle atrophy inhibitor and muscle-atrophy inhibiting composition of the present invention have an excellent muscle-atrophy inhibition effect.

Accordingly, the present invention also relates to a muscle-atrophy inhibition method including administration of at least one selected from the group consisting of the muscle atrophy inhibitor and the muscle-atrophy inhibiting composition to a subject.

### EXAMPLES

Test Examples and Formulation Examples of the present invention will be described hereinafter, but the present invention is not limited to Text Examples and Formulation Examples below.

### Test Example 1: Test for Myoblast-Proliferation Promotion Effect

A test on a myoblast proliferation effect of the compound represented by Structural Formula (1) (using the compound available from Sigma-Aldrich) was performed according to the following testing method.

### <Testing Method>

After culturing skeletal muscle-derived myoblasts (C2C12) of mice in a Dulbecco's modified eagle medium (DMEM) containing 10% FBS, the cells were collected by trypsinization. The collected cells were diluted with a 5% FBS-containing DMEM so that a concentration of the cells was to be 2.0×10⁴ cells/mL. Then, the dilution was seeded by 100 μL per well in a 96-well plate, followed by culturing for 6 hours. After completing the culture, a test sample dissolved in a 5% FBS-containing DMEM (see Table 1 below for the final concentration) was added by 100 μL per well, followed by culturing for 2 days. Even in the case where the test sample was not added, culturing was performed in the same manner except that the test sample was not added.

The myoblast-proliferation promotion effect was measured using an MTT assay. After completing the culture, the medium was removed, and MTT dissolved in a PBS buffer solution (PBS(−)) at a final concentration of 0.4 mg/mL was added by 100 μL per well. After culturing for 2 hours, blue formazan generated within the cells was extracted with 100 μL of 2-propanol. After the extraction, absorbance at a wavelength of 570 nm was measured. In addition, absorbance at a wavelength of 650 nm was measured as turbidity. The difference between the absorbance at the wavelength of 570 nm and the absorbance at the wavelength of 650 nm was used to determine an amount of blue formazan generated.

The myoblast proliferation promotion rate (%) was calculated from the obtained results according to the following equation. The results are presented in Table 1.

Myoblast proliferation promotion rate (%)=A/B×100

In the equation above, A and B are as follows.
A: An amount of blue formazan generated in the cells to which the test sample was added.
B: An amount of blue formazan generated in the cells to which the test sample was not added.

TABLE 1

| Test sample | | Myoblast |
|---|---|---|
| | Concentration | proliferation |
| Type | (μmol/L) | promotion rate (%) |
| Compound represented by | 25 | 106.4 |
| Structural Formula (1) | 100 | 108.0 |

It was confirmed from the results of Table 1 that the compound represented by Structural Formula (1) exhibited an excellent myoblast-proliferation promotion effect.

### Test Example 2: Test for Muscle Atrophy Inhibition Effect

Whether muscle atrophy was inhibited by orally administering the compound represented by Structural Formula (1) (using the compound available from Cayman Chemical) to a rat model of disuse muscle atrophy for 14 days was tested according to the following testing method.

### <Testing Method>

Seven week-old male Wistar (Crlj: WI) rats were acquired from Charles River Laboratories Japan, Inc., and were bred in the environment where a temperature was 23±3° C. and the humidity was 55±15% and in a cycle of 12 hours in light and 12 hours in dark. The rats were acclimated for one week before use, and were allowed to freely intake a solid feed (CRF-1, available from Oriental Yeast Co., Ltd.) and tap water.

After one week of acclimation, the Wistar rats were randomly divided into groups of eight. The rats were bred for 14 days in the state where the tail of each rat was held with clips so that the rat's hind legs were raised above the floor according to the method of Morey-Holton E R et al. During the breeding period, the rats were allowed to move around within a case with their forelimbs, and were allowed to freely intake water and a feed.

The test groups were (1) a normal group (without hindlimb suspension), (2) a control group (hindlimb suspension+0 mg/kg of the compound represented by Structural Formula (1)), (3) a group with the compound represented by Structural Formula (1) (hindlimb suspension+125 mg/kg of the compound represented by Structural Formula (1)).

The compound represented by Structural Formula (1) was dissolved in water, and the solution was administered by oral gavage every day for 14 days. Water was administered to the control group.

At the end of the test, the rats were euthanized by exsanguination under isoflurane anesthesia. Then, the soleus muscles were removed and the muscle weight was measured. The results are presented in Table 2. In Table 2, the soleus muscle weight is an average value.

TABLE 2

| | | | Group with compound |
|---|---|---|---|
| | | | represented by |
| | | | Structural Formula (1) |
| | | | (administration |
| | Normal group | Control group | of compound |
| | (normal | (hindlimb | represented by |
| | control) | suspension) | Structural Formula (1)) |
| Soleus muscle | 359.1 | 139.3 | 151.3 |
| weight | | | |

As presented in Table 2, it was revealed that the soleus muscle weight of the group with the compound represented by Structural Formula (1) was increased compared to the control group. It became clear from the above that the compound represented by Structural Formula (1) inhibited muscle atrophy in the rat model of disuse muscle atrophy.

### Formulation Example 1

Tablets having the following formulation were prepared according to a common practice.

The compound represented by Structural Formula (1): 5.0 mg Dolomite: 83.4 mg (including 20% of calcium and 10% of magnesium)
Casein phosphopeptide: 16.7 mg
Vitamin C: 33.4 mg
Maltitol: 136.8 mg
Collagen: 12.7 mg
Sucrose fatty acid ester: 12.0 mg

### Formulation Example 2

An oral liquid preparation having the following formulation was prepared according to a common practice.

### <Formulation for One Ampule (100 mL Per Ampule)>

The compound represented by Structural Formula (1): 0.3% by mass
Sorbitol: 12.0% by mass
Sodium benzoate: 0.1% by mass
Fragrance: 1.0% by mass
Calcium sulfate: 0.5% by mass
Purified water: balance

### Formulation Example 3

A coffee drink having the following formulation was prepared according to a common practice.

The compound represented by Structural Formula (1): 0.1% by mass Coffee extract: 40% by mass (L (brightness)=20, Brix=3)
Maltitol: 2% by mass
Fragrance: appropriate amount
Water: balance

### Formulation Example 4

Capsules having the following formulation were prepared according to a common practice. As capsules, hard gelatin No. 1 capsules were used.

### <Composition in 1 Capsule (200 mg Per Capsule)>

The compound represented by Structural Formula (1): 30.0 mg
Corn starch: 70.0 mg
Lactose: 80.0 mg
Calcium lactate: 10.0 mg
Hydroxypropyl cellulose (HPC-L): 10.0 mg

The present application claims priority based on Japanese Patent Application No. 2022-095282, filed on Jun. 13, 2022, the entire content of which are incorporated herein by reference.

1. A myoblast proliferation promoter, comprising:
a compound represented by Structural Formula (1) below,

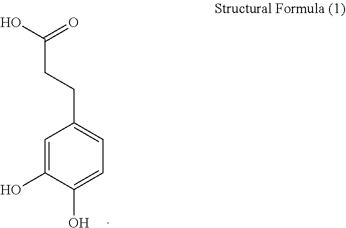 [SMILES: O=C(O)CCc1ccc(O)c(O)c1]

2. A myoblast-proliferation promoting composition, comprising:
the myoblast proliferation promoter according to claim 1.

3. A muscle atrophy inhibitor, comprising:
a compound represented by Structural Formula (1) below,

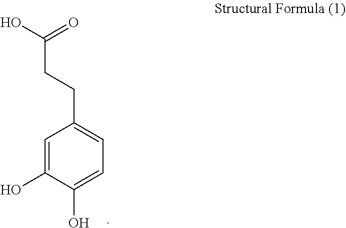 [SMILES: O=C(O)CCc1ccc(O)c(O)c1]

4. A muscle-atrophy inhibiting composition, comprising:
the muscle atrophy inhibitor according to claim 3.
